(19) United States
(12) Patent Application Publication
(10) Pub. No.: US 2023/0240581 A1
(43) Pub. Date: Aug. 3, 2023

(54) SYSTEM AND METHODS FOR BIOSIGNAL DETECTION AND ACTIVE NOISE CANCELLATION

(71) Applicant: University of Central Florida Research Foundation, Inc., Orlando, FL (US)

(72) Inventor: Seyed Yahya Shirazi, New York, NY (US)

(21) Appl. No.: 18/104,651

(22) Filed: Feb. 1, 2023

Related U.S. Application Data
(60) Provisional application No. 63/306,034, filed on Feb. 2, 2022.

(51) Int. Cl. A61B 5/291; A61B 5/31; A61B 5/00; A61B 5/259

(52) U.S. Cl. CPC A61B 5/291; A61B 5/31; A61B 5/7203; A61B 5/259

(57) ABSTRACT

An apparatus for sensing electrical currents in a subject has a geodesic net structure of electrode elements connect by flexible legs. The electrode elements each have an inner electrode facing and sensing electrical currents in the subject and an outer layer electrode facing away and sensing external electrical noise. The legs have flexible conductive material that electrically connects the outer electrodes so that they are all connected and are electrically the same or similar to the subject's body part. The outputs of the electrodes are converted to multiplexed digital signals and transmitted to signal processing circuitry that identifies the noise present in the signals from the outer electrodes and removes the noise from the signals from the inner electrodes so as to output clean EEG data for each inner electrode. Additional electrodes that detect extraneous neuro-muscular currents are also used to determine the noise in the inner electrode output signals.

### RELATED APPLICATIONS

This application claims the priority of U.S. provisional patent application Ser. No. 63/306,034 filed Feb. 2, 2022, which is herein incorporated in its entirety by reference.

### FIELD OF THE INVENTION

This invention relates to systems and methods of sensing electrical signals produced by the body of a patient, and, more particularly, to systems and methods for taking electroencephalograms from head-mounted detection systems.

### BACKGROUND OF THE INVENTION

The human body as part of its neurological activity produces a number of electrical currents that may be detected for medical purposes. Surface electrophysiological recordings (EXG) include many modalities of non-invasively bodily electrical signals that are used to analyze the body responses to internal and external inputs and interactions.

Surface electroencephalography (EEG), Electromyography (EMG), Electrooculography (EOG), electrocardiography (ECG), and, in general, EXG are methods to record brain, muscle, heart, eye-movement, and other biological electrical signals at the skin with non-invasive electrodes. The relatively easy and fast setup, non-invasiveness, portability, and affordable cost of EXG have made this approach extremely popular to study brain and body dynamics in various research and clinical settings. In the past decade, the interest in EXG as a method of interaction between mind/body and machine has been dramatically increased, but technical barriers such as susceptibility to noise hindered widespread use of EXG in daily life.

EEG records brain electrical activity from the scalp of the person. Some biosignals, such as the brain electrical signals, have very low amplitude, i.e., at microvolt level, at the scalp, and a slew of artifacts originating from the person or the environment can easily overshadow those biosignals. While EEG offers non-invasive, mobile, and low-latency brain monitoring, EEG's susceptibility to artifacts is a long-standing challenge to use of this method.

Interfering signals from the person include unwanted muscular activity, especially in the neck and face areas, heart electrical signals, and eye movement. Interfering signals from the environment come from the physical movement of the EXG cables and sensors relative to each other, and from outside electrical sources such as fluorescent lamps, electromotors, or even power lines. Current best practices for acquiring EEG (and EXG in general) therefore are to limit head and body movements as much as possible, and to conduct the test in an electrically isolated environment.

One way that has been tried to overcome the noise problems is a dual-layer EEG, in which both brain signals from the scalp and the environmental artifacts from a second-layer conductive fabric isolated from the scalp are simultaneously recorded. The second layer has electrodes that are mechanically coupled to the EEG electrodes in contact with the scalp, and movement artifacts, environment artifacts, and even artifacts due to cable sway are presumed to be similar between the EEG electrodes and the second layer electrodes. However, data from the second layer has been only used during post-processing (i.e., after data is collected and stored), not in real-time. Also, other sources of artifacts, such as muscle activities in the neck or eye areas, are not included in the second-layer data.

In addition, the dual-layer EEG setup is a very time-consuming and delicate process, requiring multiple training sessions, and is much less comfortable for the subject because of the extra pressure from the second layer, which is usually very confining and tight around the subject's head.

Hardware and signal processing have also been used to try to address the abundance of noise, especially in EEG. Examples of hardware systems used include recording concurrent recording of muscular and ocular activity, EEG common-mode rejection, and recording environment noise with dual-layer EEG. Signal processing improvements mainly include separating noise from what is presumably “brain” data using mathematical methods such as artifact subspace reconstruction, (see, e.g., P. Anders et al., “The Influence of Motor Tasks and Cut-off Parameter Selection on Artifact Subspace Reconstruction in EEG Recordings”, Medical & Biological Engineering & Computing, 58:2673-2683, doi: 10.1007/s11517-020-02252-3, Aug. 28, 2020; see also, S. Blum et al., “A Riemannian Modification of Artifact Subspace Reconstruction for EEG Artifact Handling”, Frontiers in Human Neuroscience, vol. 13, page 141, doi:10.3389/fnhum.2019.00141, Apr. 26, 2019), principal component analysis (see, e.g., U. Acharya et al., “Use of Principal Component Analysis for Automatic Classification of Epileptic EEG Activities in Wavelet Framework”, Expert Systems with Applications, vol. 39, issue 10, pages 9072-78, August 2012, doi: 10.1016/j.eswa.2012.02.040; see also A. Delorme et al., “Independent EEG Sources Are Dipolar”, PloS One, vol. 7, issue 2: e30135, Feb. 15, 2012), independent component analysis (see, e.g., J. Palmer et al., “Modeling and Estimation of Dependent Subspaces with Non-Radially Symmetric and Skewed Densities”, in M. E. Davies et al., Independent Component Analysis and Signal Separation, ICA 2007, Lecture Notes in Computer Science, vol. 4666, Springer, Berlin, Heidelberg, doi: 10.1007/978-3-540-74494-8_13, 2007); J. Palmer et al., “Newton Method for the ICA Mixture Model”, in 2008 IEEE International Conference on Acoustics, Speech and Signal Processing, pp. 1805-1808, doi: 10.1109/ICASSP.2008.4517982, 2008), empirical mode decomposition (see, e.g., K. Al-Subari et al., “EMDLAB: A Toolbox for Analysis of Single-Trial EEG Dynamics Using Empirical Mode Decomposition.”, Journal of Neuroscience Methods, vol. 253, pages 193-205, DOI: 10.1016/j.jneumeth.2015.06.020, July 8 and Sep. 30, 2015), and canonical correlation analysis (see, e.g., A. Janani et al., “Improved Artefact Removal from EEG Using Canonical Correlation Analysis and Spectral Slope”, Journal of Neuroscience Methods, vol. 298, pages 1-15, DOI: 10.1016/j.jneumeth.2018.01.004, February 4 and Mar. 15, 2018. Those methods, however, are still subject to the problem of noise overcoming the brain data signals.

Another issue with the prior art approaches is that attempts to improve signal quality usually come at the expense of the subject's comfort and the preparation time of the output. The signal processing approach has the drawback that there is an issue as to the accuracy of the presumed “brain” data and the high computational cost of separating noise from the biosignal in real-time.

### SUMMARY OF THE INVENTION

It is accordingly an object of the invention to provide a system for deriving an EXG output corresponding to one or more biosignals of a patient that avoids the problems of the prior art. EXG signals have different levels of susceptibility to noise, but, because surface electroencephalography (EEG) is one of the most sensitive signals to outside noise, the invention is particularly applicable to EEGs. Nonetheless, the system and methods described here are easily transferrable to other methods of biosignal recording modalities as well.

According to an aspect of the invention, an apparatus for sensing biosignals of a head of a subject comprises a net structure configured to be supported on the head of the subject. The net structure comprises a plurality of electrode structures connected in the net structure by elastic legs each connected with a respective pair of the electrode structures. The electrode structures each include a respective first electrode directed toward and sensing biosignals in the head of the subject, and a respective second electrode supported adjacent the first electrode and directed away from the head of the subject and sensing electrical signals in an environment around the head of the subject. The legs each have a respective elastic conduction element extending between the associated electrode structures. The conduction elements are connected electrically with the second electrodes of the electrode structures connected with the leg. Elastic insulation structures are between the conduction elements and the head of the user so as to electrically insulate the conduction elements from the head of the user.

The legs also each preferably have an outwardly disposed elastic insulation layer outward of the elastic conduction elements. The net structure is preferably a geodesic arrangement in which some of the electrode elements are connected with five legs or six legs, all of which have their conduction elements connected with the second electrode so that the second electrodes of the net structure are all interconnected electrically, and so that the net structure has electrical properties that are similar to, or the same as, electrical properties of the head of the user. By this is meant similar resistance and conductivity such that both the skin of the subject and the layer experience similar or the same electrical signals due to external electrical influences. The net structure then also has electrode elements with first and second electrodes at a perimeter of the net structure. Those further electrode elements have only two, or fewer than five, links to adjacent electrode structures of the net structure.

According to another aspect of the invention, the electrode structures each have a respective analog-to-digital converter that receives analog electrical signals from the first and second electrodes, and converts them to digital signals that are output to digital circuitry that processes the digital signals so as to derive EEG data therefrom. The electrode structures also preferably each have a multiplexer that receives raw signals from the first and second electrodes and multiplexes those raw signals with a control signal having a frequency of 5 kHz or greater, and transmits a resulting multiplexed output to the analog/digital converter. In the preferred embodiment, the two varying-amplitude analog signals are combined by a multiplexor using the high frequency control signal to produce a single analog output signal in which the content of the signal alternates between one electrode and the other in alternate cycles of the control signal. That multiplexed analog signal is then transmitted to an analog-digital converter that converts the amplitude of each cycle to a respective digital data packet that contains a digital value in 2 to 4 bytes that corresponds to the amplitude in that signal and a digital time stamp. The data packets are transmitted sequentially as a single combined multiplexed digital signal from the electrode unit that carries the data of both of the analog channels to the signal analysis electronics. The multiplexer alleviates the need for separate analog to digital converters for the first and the second layers and makes a single multiplexed digital output for the two signals that is demultiplexed (i.e., sequential data packets are separated and their data stored and processed) downstream so as to recover the data of the amplitudes of both the first and second electrodes. Based on the usage of the device, the multiplexer can operate at varied frequencies but needs to accommodate broadband EEG recordings.

The digital circuitry processes the digital signals by dividing the signals from the first and second electrodes into constituent components. The circuitry, which may be a computer system or a dedicated circuit, then identifies those distinct components of the signals from the first electrodes that are not present in the components of the signals from the second electrodes. Those distinct components are then output as the EEG data.

The apparatus may further comprise additional electrodes that sense electrical currents of the subject related to muscle activity of the subject, such as of the eyes or other muscles on the head of the subject. Output from the additional electrodes is combined with the signals from the second electrodes to form a combined noise signal prior to dividing the signals into constituent components.

The constituent components may be derived by a variety of methods known in the art for separating signals into separate waveforms. In the simplest sense, the signal may be divided into sets of frequencies in a Fourier breakdown of the signal, in which case individual frequencies or ranges of frequencies are isolated from the composite noise signal. Alternatively, methods of identifying component waveforms are employed that differentiate waveforms over time, such as independent component analysis. In that type of analysis, waveforms are differentiated by their different presence over the time domain, meaning that waveforms with the most mutual statistical independence are separated from each other in the composite noise signal.

Additionally, the noise signal may be divided into component signals that are identified as biologically implausible, i.e., signals that could not be reasonably expected to be actual biosignal data received from a person being monitored in the given application.

The noise signal may further be also divided into components using the same or different methods applied to the composite signals, including principal component analysis (PCA), independent component analysis, empirical mode decomposition, canonical correlation analysis, or another method of dividing up a composite signal into constituent waveforms of which it is comprised.

Once the noise signals are divided into their constituent components, those components are removed from, or disregarded in, the combined noise and biosignal data signal from the inward-facing first electrodes, leaving the biosignal without noise to be output as the EEG of the subject, on a display or printout attached to the system, or as data that can be accessed and stored by a computer of the system.

According to another aspect of the invention, a method of sensing electrical currents in the skin of a subject comprises deriving outputs from first electrodes directed toward the skin of the subject, and deriving outputs from second electrodes each connected with a respective first electrode and directed away from the skin of the subject in an electrically connected net structure that has electrical properties similar to electrical properties of the skin of the subject. The method then includes dividing each of the outputs into a number of discrete components each of which has respective frequency or waveform characteristics. The discrete components are then clustered so as to identify the discrete components that are present in outputs from the first electrodes (i.e., the electrodes picking up the biosignals mixed with environmental noise) but not in outputs from the second electrodes (the electrodes that are insulated from the subject and thus pick up primarily the environmental noise). Those identified discrete components are then output or recorded as sensed electrical currents in the associated body part of the subject. The entire method is preferably conducted in real-time.

In addition, signals may be derived from electrodes picking up electrical background currents created by muscle activity in the subject, such as eye movement, neck movement, or other neuromuscular noise. Those signals are then scaled to correspond in amplitude to amplitudes of the signals from the second electrodes over a period of the previous few milliseconds prior, and combined with the signals from the second electrodes prior to dividing the outputs into the discrete components.

The method is most preferably used for an EEG, with the skin of the subject being on a head of the subject on which the first electrodes are placed with conductive gel between them. However, other applications are possible where biosignals from other parts of the body are to be detected.

According to aspects of the invention, improvements are provided that are directed to hardware and electronic signal processing with a computer operating on stored software instructions or dedicated signal processing circuitry that improve EEG (and EXG in general) hardware comfort, eliminate the presumptions for brain data, and that reduce computational overhead. On the hardware level, dual-layer EEG principles (the gold standard for EEG data collection during movement) are integrated into a comfortable EEG system. Currently, the dual-layer approach is used solely at the research level, and the setup process for its use is time-consuming and uncomfortable for both experimenters and subjects. The known setup requires putting an EEG cap and electrodes (both brain-facing and environment-facing electrodes) on the subject's head, and then next putting a conductive fabric as the second layer on top of that, and then applying gel to make a connection between the environment-facing electrodes and the conductive fabric.

Avoiding that, the hardware apparatus of the invention in one aspect has a second layer and the cap blended together, and the placement of sensors on the cap is complete before putting the cap on the subject's head. This approach improves the usability of the dual-layer EEG, significantly reducing the setup time, and adding to the apparatus robustness because fewer pieces need to interact with each other.

Another aspect of the invention is to avoid artifacts from cable movements, which are also a source of noise, by circuitry that practically eliminates this source of noise completely by introducing analog to digital converters at each electrode unit, i.e., each element with an inner and an outer electrode.

According to still another aspect of the invention, processing EEG (and EXG in general) signals employs three methods for 1) referencing, 2) immersive noise inclusion, and 3) computationally-affordable noise separation. The referencing method takes the local and/or global noise environment into account, as will be described herein. Methods for including multiple sources of noise together create an immersive noise layer in real-time. This virtual-noise data layer provides a real-world baseline for the noise content in real-time and separates noise from useful information, i.e., biosignals that are mixed with the noise. The method also uses fast noise-rejection methods to get the noise content in real-time and to separate clean signals from noise.

In combination, these methods significantly improve high-quality EEG and EXG data for industries and researchers outside neuroscience laboratories. They also improve EXG portability, making brain and body signal recording possible for rehabilitation, autonomous driving, and many other brain-computer interactions. Also, because noise features are analyzed and separated in real-time, the quality of the EEG and EXG data is increased in mobile settings, which, in return, provide more useful information about human decisions and responses to their environment.

Both the processing computer and software system (or dedicated signal processing circuitry) and the apparatus hardware design make the dual-layer EEG setup easier, reduce the sources of artifacts and process the dual-layer data in real-time. The processing includes:
1) a real-time method that includes muscular activity EMG in the noise data;
2) real-world referencing, meaning a method for referencing the EEG layer based on the second layer or based on the immersive noise layer (the terms “second-layer” and “noise-layer” are used interchangeably herein); and
3) the reverse artifact subspace reconstruction (ASR) artifact subtraction, which uses readily available and real-time signal separation algorithms in reverse order to separate the noise from EEG biodata signals.

The hardware innovations include:
1) a digital dual-layer concurrent EEG (DDLC-EEG) system that transmits signals from both the first-layer EEG electrodes and second-layer electrodes coupled with an analog-to-digital converter inside the electrode enclosure, and
2) a geodesic dual-layer EEG sensor structure that provides an EEG system that includes the second noise layer.

Output from an EEG is a combination of brain signals, artifacts from the body, muscle activity, and environmental artifacts. The more information known about the artifacts, the better the chances of cleansing the EEG of artifacts. In the method disclosed herein, the EMG signals from the neck and eye muscles are mixed with the second-layer data to integrate the artifact sources. This method is referred to as the immersive noise layer.

EMG amplitude can be multiples greater than environmental noise. To prevent this difference from overshadowing the noise characteristics, an additional monitoring layer compares the EMG amplitude with the noise level in the past several milliseconds, and adjusts the EMG amplitude before mixing the signals.

Like all other electrical signals, EEG signals are measured against a reference. Ideally, the reference should be electrically neutral, meaning that there should be minimal meaningful electrical activity at the reference location. Currently, the most common references are the mastoids, common-average reference, and the single-electrode reference. Those references can partially reduce the large-amplitude and global noise but fail to address the local contamination, including cable sway, eye movements, muscle activity, and electrode movements. Also, since all commonly-used references partially include brain signals, some brain signals will be lost using these references.

In the present system and methods, the second-layer is used as the reference for the EEG. Because the second layer is electrically isolated from the scalp, it does not contain any brain signals. Also, having the second-layer as the reference results in canceling the noise signals captured in the second layer from EEG. This approach is referred to as real-world EEG referencing. This sort of referencing can be used with only the second-layer or the immersive noise-layer. Alternatively, the referencing can be performed in a bipolar way, that is, each of the first and second layer electrodes are referenced to a common-mode reference with or across the layers, and then the signals from the first layer are referenced or compared to the signals of the second layer.

An alternate approach of the invention is to reference the output of each EEG electrode to output from its second-layer counterpart, which results in localized noise cancellation.

Another feature of an aspect of the invention is reverse ASR. Artifact subspace reconstruction (ASR) is a fast computational tool that separates the EEG signals from noise. This method, however, requires prior training of the system so that it can learn the “noise-free” EEG characteristics, so that, in the presence of noise, the system can separate EEG from the noise.

Acquiring “noise-free” EEG is to a degree variable and subjective to each person and task. For example, for recording EEG during driving a vehicle, the “noise-free” scenario would be having each person drive on a simulator for few minutes with similar street and environment conditions as the real-work experiment to characterize most of their EEG characteristics. Then in the real-world scenario, ASR picks the EEG portions of the data and rejects the noise data.

According to an aspect of the invention, ASR is used in reverse order, that is, by first training ASR with the noise-layer data so as to identify the noise signals, and then separating the noise from the EEG data, leaving cleaner EEG data. Since real-time information about noise-data is available from the second layer, ASR is provided with real-time information about the noise characteristics and that method is used to separate noise from EEG. As a byproduct, a clean EEG is found in the rejected data in the ASR pipeline.

Traditional ASR applies a computational method to the combined EEG data signal and noise from the individual being monitored, and extracts a separate noise-free EEG data signal from that composite signal. In the present invention, in contrast, reverse ASR use any source separation algorithm, including but not limited to principal component analysis, independent component analysis, empirical mode decomposition, canonical correlation analysis, non-negative matrix factorization, and singular value decomposition, and identifies components in the pure noise signal from the outer layer of electrodes. Once those noise components are identified, then those noise components from the second layer are removed from or disregarded in the composite EEG and noise signal from the inner layer of electrodes, resulting in the clean EEG output.

According to another aspect of the invention, the digital dual-layer concurrent EEG (DDLC-EEG) provides an advantage by converting the analog EEG and second-layer signals to digital inside the electrode enclosure. This eliminates cable movement and mass, which are one of the main sources of artifacts, and makes the overall EEG enclosure more portable because the analog to digital conversion is distributed to each electrode instead of concentrated on a central module.

Having the EEG and second-layer EEG signals converted at the same time also increases the synchronization of the data and improves using the second layer as the reference for EEG. A Digital Signal Hub (DSH) that receives the signals both collects the digital signals and provides the steady supply voltage and clock to sync the electrodes with each other.

The Geodesic dual-layer EEG also implements the second layer on top of a geodesic EEG system to improve the portability, convenience, and robustness of the dual-layer EEG. Instead of putting a conductive cap as the second layer, which puts excessive pressure on the head and obscures the access to the electrodes during the experiment, conductive fabric is routed between and within the geodesic links. The conductive fabric covers the second-layer electrodes, and the user can conveniently inject conductive gel between the second-layer electrode and the conductive fabric to connect them electrically. This electrical connection can also be maintained by protruding each of the second layer electrodes with a dome-shaped design, so that it always is in contact with the conductive fabric. The conductive fabric net is connected with all of the electrodes of the apparatus, and is of material and dimensions that are selected so that the conductive fabric net electrically emulates the electrical properties of the scalp of the person being given the EEG.

Other features and advantages of the invention will become apparent to one of skill in the art from this specification.

### BRIEF DESCRIPTION OF THE DRAWINGS

FIG. 1 is a diagram showing single- and dual-layer EEG electrodes of the prior art.

FIG. 2 shows an assembly of the prior art dual-layer EEG headgear.

FIG. 3 is a diagram showing a geodesic dual-layer EEG head-mounted apparatus according to the invention.

FIG. 4 is a diagram showing a side view of a dual electrode unit of the nodes of the geodesic EEG apparatus of FIG. 3.

FIG. 5 is a plan view of the electrode unit with connected legs of the geodesic apparatus of FIG. 3.

FIG. 6 is a diagram of the enclosure of the electrode unit of FIGS. 4 and 5.

FIG. 7 is a schematic diagram of the electronic arrangement of the electrode units of the geodesic apparatus of FIG. 3.

FIG. 8 is a diagram showing the combination of the EMG noise data with the environmental second-layer noise data.

FIG. 9 is a diagram of the process of conditioning or scaling the EMG muscle signals to the amplitude of the noise (second) layer.

FIG. 10 is a diagram of the signal processing and noise reduction of an embodiment of a system of the present invention.

FIG. 11 is a diagram showing the prior-art methods for referencing EEG signal output.

FIG. 12 is a diagram showing a method for removing noise from an EEG signal.

FIG. 13 is a diagram showing another method for removing noise from an EEG signal.

FIG. 14 is a diagram showing still another method for removing noise from the EEG signal.

### DETAILED DESCRIPTION

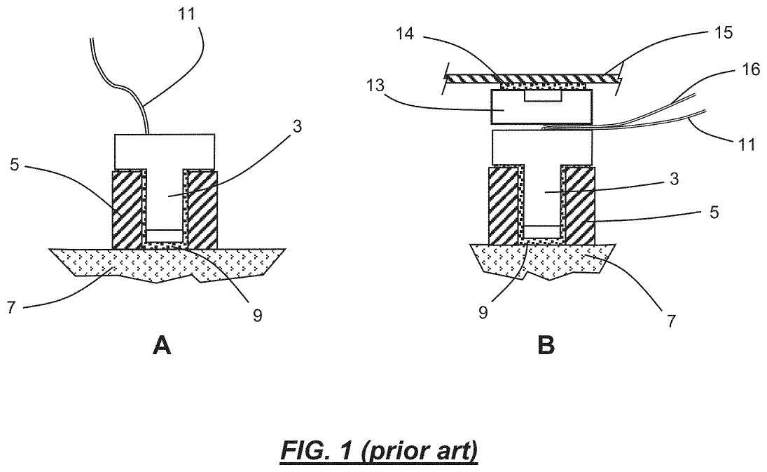

FIG. 1 shows single-layer and dual-layer EEG electrodes of the prior art, which are electrodes that may be employed in the present invention.

In FIG. 1(A), a single-layer pin-electrode EEG 3 fits into the EEG-cap well 5 and has indirect contact with the skull 7 via a conductive gel 9 in the well 5. The EEG biosignals are carried to associated electronics via wire 11. In FIG. 1(B), a dual-layer EEG electrode includes the single-layer EEG pin-electrode 3 of A to record signals from the scalp 7 (the first layer), and it further includes a flat electrode 13 facing toward the environment and away from the skull to record noise (the second layer). To make the second layer work, a second-layer conductive fabric 15 with electrical characteristics similar to the scalp covers and surrounds the outer noise electrodes, and acts as an artificial scalp to provide the necessary resemblance between the first and second layer. See A. Nordin, “Dual-Electrode Motion Artifact Cancellation for Mobile Electroencephalography”, Journal of Neural Engineering 15 (5): 056024 (2018). The conducting fabric 15 is electrically connected with the noise electrode 13 by conductive gel 14. The electrode 13 outputs the second-layer noise signal by output wire 16.

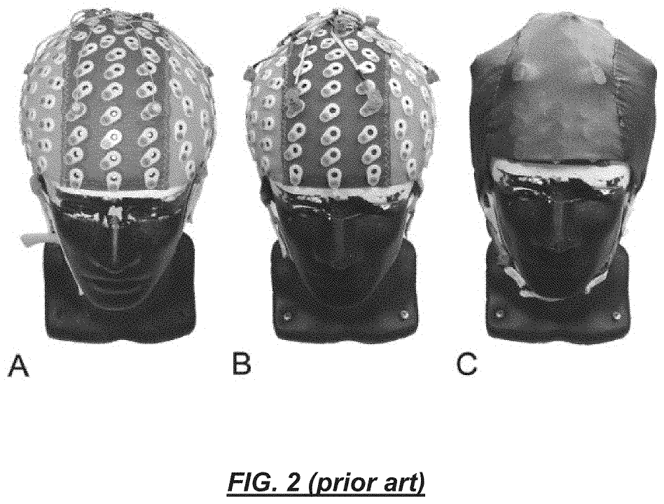

Existing systems that provide these dual-layer electrode arrangements are very cumbersome and difficult to install. A prior-art EEG head-mounted dual-layer apparatus is shown in FIG. 2. Placing the apparatus on the head of the subject is a multistep procedure of putting the inner electrode headgear on the subject, then adding the outer layer electrodes, and then affixing the conductive outer fabric outside of that.

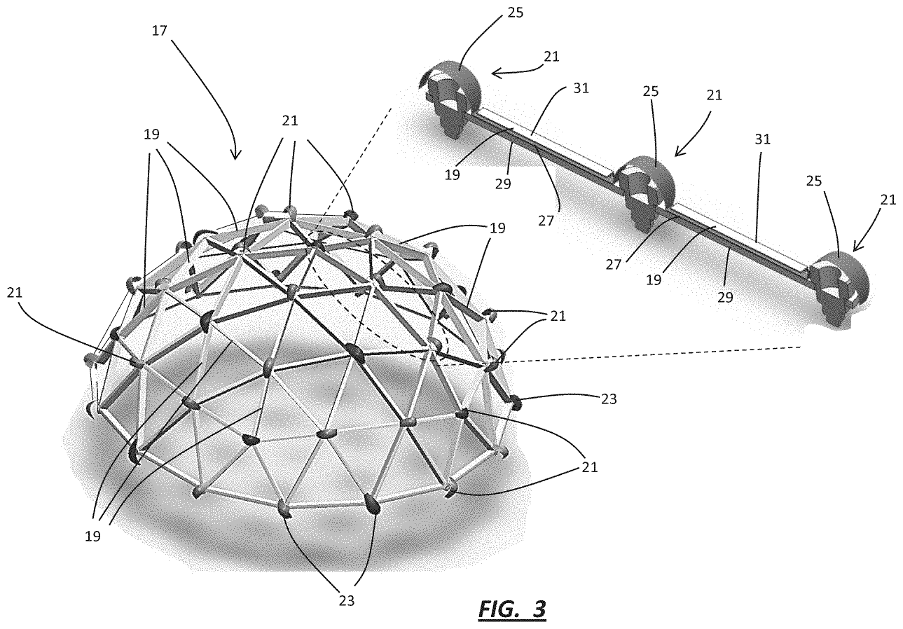

The headgear apparatus of the present invention avoids this complicated procedure, and is shown in FIG. 3. The headgear is a geodesic net 17 that fits over the head of the subject. The net 17 is made up of elastic links or legs 19 connecting electrode units 21, each of which is at a node of a geodesic net pattern made up of triangular link structures organized as hexagons or pentagons around each electrode unit node, except for the units on the edge of the headgear 17, e.g., electrode units 23. Each electrode unit 21 or 23 is made up of an inward-facing electrode facing the subject, and a second-layer noise electrode facing away from the subject as will be set out herein.

The legs are made of nonconductive elastomeric polymer material such as is used in single-layer geodesic EEG headgear, and allow for stretching of the net 17 to fit onto the subject's head.

In addition to the nonconductive elastic material, the legs 19 also include strips of elastic conductive material 27 that overlie a lower layer 29 of the nonconductive leg material, and that are, in turn, covered by a protective layer of nonconducting elastic material 31. The conductive material also has portions indicated at 25 that overlie and are electrically connected with the outwardly disposed second layer electrodes. The conductive material of all the legs is preferably cut from a single sheet of material, and together the legs 19 provide a structure electrically linking all of the outward second layer electrodes that provides a structure with electrical properties similar to the scalp so as to receive environmental signals in the same way so that the noise can be detected separately from the EEG data in the scalp.

The geodesic dual-layer EEG relies on the second layer being an elastic and conductive material or a like fabric that stretches above the noise portion of the dual-electrode. The fabric runs between the electrodes, creating the second conductive layer. To prevent contacting the second layer with the skin, or other unwanted contact with the subject, the second layer runs in a sandwich of the nonconducting elastic connections between the electrodes.

The elastic conductive material is preferably the material sold by Eeonyx Corp, Pinole, Calif., under the trade name Eeyonyx. It has electrical impedance or resistance that is between 0.1 and 1 MOhm over the length of the legs 19. The dimensions of the strips of material in legs 19 are preferably a width of at least 5 mm, a thickness of less than 5 mm, preferably 2 mm to 3 mm, and a length between the electrode units of greater than 1 cm, preferably 2 cm to 4 cm between the electrode units of the geodesic net.

The outer protective layer 31 of elastomeric nonconductive material surrounds the conductive layer 27 and protects it from possible electrical contacts or other artifacts from contact outside the subject.

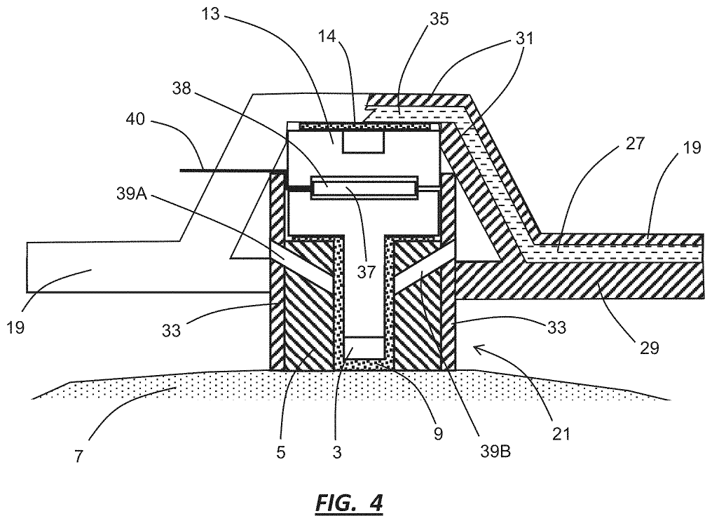

FIG. 4 shows in detail the structure of each of the electrode units 21 and its associated legs 19. The parts of the electrode unit 21 that it has in common with the dual layer structure of FIG. 1(B) are given the same reference numbers, i.e., skull 7, inner electrode 3, outer electrode 13, cap well 5, and conductive gel 9, 14.

In the electrode unit 21, first layer electrode 3 and its cap well 5, and second layer electrode 13 are both supported in an enclosure or housing 33 that is securely connected with ends of the links or legs 19 supporting the electrode unit 21. The housing 33 is connected with the supporting nonconductive elastic layer 29. The conductive layer 27 overlies that layer 29, and extends upward surrounded by the protective insulation of layer 31 up to an upper part of the electrode unit 21, where a portion 35 of the inner (and, if needed, outer) conductive layer 27 is exposed, and is electrically connected with the outer second-layer electrode 13 by gel 14, linking the electrode 13 electrically to the entire net. The conductive fabric completely covers the second-layer electrode, and is connected electrically to all of the conductive layers 27 of all of the legs 19 running to that particular node of the EEG geodesic network. The outer layer 31 completely overlies and insulates the outer surface of the central portion 35 of the conductive layer. The gel may be applied between the interface of the electrodes and the scalp or the conductive fabric in several ways, including the use of a saline-soaked foam or a semi-permeable hydrogel, commonly referred to as “dry gel”, or by injecting conductive gels at the interface using a syringe. To facilitate this, the electrode unit 21 has conduits 39A and 39B communicating from the exterior of the headset to the space around the electrode on the skin of the subject. One of these channels 39A or 39B can be used as an access passage into which a syringe may be inserted, allowing injection of the gel, and the other as a vent that permits escape of any air displaced by the injection of the gel.

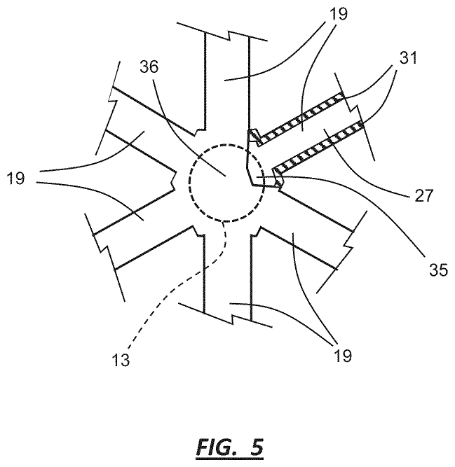

FIG. 5 shows the plan view of the electrode unit 21 with a part of the outer layer 31 cut away to show the conductive layer 27. All the links 19 have a conductive layer 27 and all are connected to the central portion 35 overlying and electrically connected with the electrode 13. The conductive material of layers 27 and central portion 35 are covered and insulated electrically by the outer layer 31 of the legs 19 and by an electrode covering central portion 36 connected with all of the associated legs 19.

It will be understood that a similar structure with five legs is present at each geodesic node that has only five legs attached, or at the edge nodes that have fewer legs.

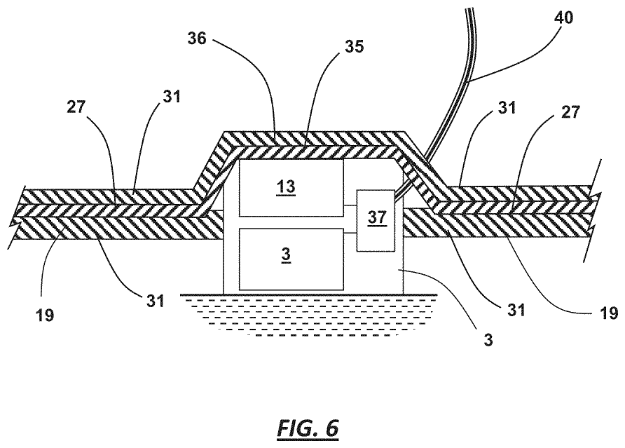

Referring to FIGS. 4 and 6, the electrode unit 21 includes housing 33 connected with links 19, and supporting in it the inner layer electrode 3 and the outer layer electrode 13. The electrically conductive net 27 is connected with the outer electrode 13 as discussed above.

As seen in FIG. 4, electrical circuitry 37 is supported in the electrode unit 21 as a small (less than 5 mm) circuit board 38 embedded in a space at the interface of the first layer electrode 3 and the second layer electrode 13, and that circuitry 38 receives the outputs from the electrodes 3 and 13 and converts them to multiplexed digital signals that are output via a single output wire 40 to the electronic system that processes the EEG output, which may be a dedicated EEG processor or a computer system programmed appropriately to perform the signal processing and output the results to be viewed by a user or used otherwise. The conversion to digital signals is a benefit of the system, because it avoids output of raw signal data through wires adjacent the electrodes, which may contribute to noise picked up by the electrodes.

Wire 40 preferably also includes additional separate wires that provide (1) constant voltage DC-current electrical power to the circuit 38 to power the A/D converter and multiplexer, and any other components in it, and (2) a connection to ground for the circuit 38.

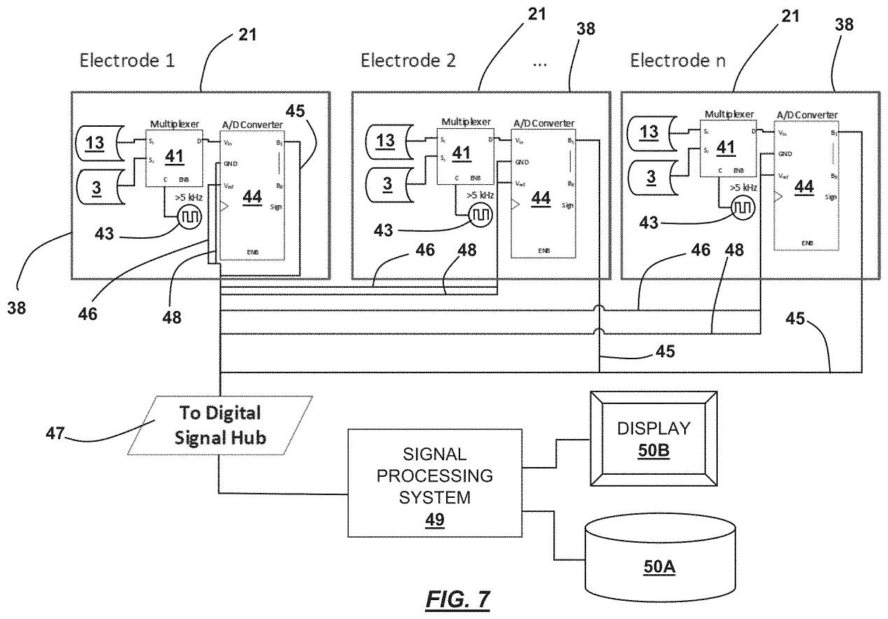

Referring to FIG. 7, each electrode unit 21 has a first layer electrode 3 and a second noise layer electrode 13 with outputs that supply the electrical signals detected by the electrodes to a multiplexer 41 in the unit circuitry 37.

Multiplexer 41 creates a sort of time division multiplex signal relying on a control signal 43 that is 5 kHz or greater, and outputs the multiplexed signal to A/D converter 44. The multiplexor produces an analog output signal created by switching the input signal between the first electrode output and the second electrode output every cycle of the control signal, which is typically a much higher frequency than any component of the electrode signal. The analog signal that is output consequently is made up of alternate cycles in which the output of the first electrode is transmitted and then the output of the second electrode is transmitted as the analog output signal. This results in loss of alternate portions of the analog output of each electrode analog signal, but the frequency of the control signal is so much higher than the frequency content of the electrode signals, e.g., greater than 3 kHz or 5 kHz, that this is not a significant loss of information. However, the combination or multiplexing of the two signals into a single analog signal allows for use of only one A/D converter for both electrodes. A/D converter 44 converts the combined multiplexed analog signal to a digital signal comprised of a series of sequential digital data packets of 2 to 4 bytes of bits at a voltage, e.g., 3 or 5 volts, each derived from a respective cycle of the combined analog signal and either corresponding to the amplitude in the cycle of either the first or second electrodes 3 and 13, and a time stamp for the digital data. A/D converter 44 outputs the digital signal along output wire 45 to electronics of digital signal hub 47, where the digital signal is demodulated into separate data for the biodata electrode and noise layer electrode signals, which are transmitted to processing circuitry or computer 48. Processing circuitry 48 processes the signals so as to remove the noise and derive a clean EEG data signal, and then stores the EEG data in data storage 50A, e.g., a computer accessible memory, and/or displays the EEG data to a user interface 50B, e.g., a display monitor. This digital dual-layer concurrent EEG (DDLC-EEG) system provides analog to digital conversion of both EEG and noise layer in the electrode enclosure, and only digital signal transfers to the Digital Signal Hub. This eliminates cable sway artifacts, which are one of the most prominent sources of EEG noise.

In addition, as described above, wires 46 providing connection to ground and wires 48 providing DC current power extend together with, alongside and electrically insulated from the data lines 45 to the digital signal hub where they connect to ground or a DC power source.

Particularly preferred as components for this circuitry are the data acquisition systems in single integrated circuits (ICs) that integrate the multiplexer, the oscillating control signal, analog to digital converter and digital signal transmission protocols. Examples of such ICs are ADS112C04 and/or ADS1115 from Texas Instruments. Both of those ICs are approximately 3 mm wide and approximately 3 mm in height, and approximately 1 mm thick, and are specifically designed for biosignal monitoring. Additionally, both chips include a virtual reference that makes the recordings of the first and second electrodes against a stable potential, adding to the consistency of the recordings.

In addition to the noise from the environment, there is also noise in an EEG signal from the muscular activity of the subject being tested. Usually this is noise from the movements of the subject's eyes or neck muscles, or other neuro-muscular sources, all collectively referred to here as EMG noise. Much of that noise may be picked up by electrodes properly placed on the subject's body that are not part of the net structure 17.

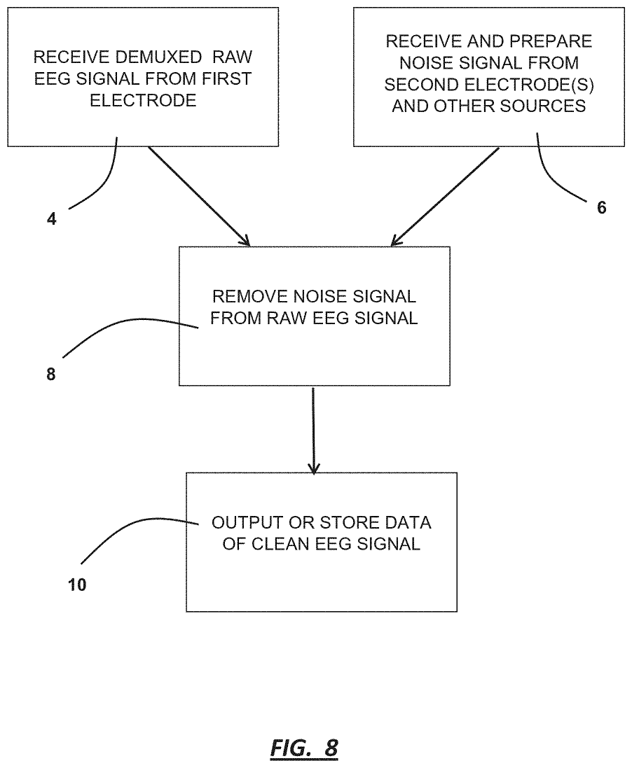

FIG. 8 shows the general operation of the signal processing system 48. The signal hub 47 transmits to the signal processor 49 digital data represents a set of data defining, for each of the inner-layer electrodes 3, a respective time-varying raw EEG signal from the subject, and also defining for each of the second-layer electrodes 13, a respective time-varying raw noise signal. The signal processor 49 receives the raw EEG data signal (step 4), and also receives the noise signals from the second-layer electrodes 13 and any other electrodes in the system that provide data that is relevant to the noise in the system, e.g., electrodes detecting neuro-muscular signals from the eyes, face, neck, mastoids, bone-reference electrodes, etc. (step 6). The signal processor 49 processes the noise signal in a variety of ways that will be described below, but generally include scaling the signals so that a comparative use of the noise signals can be made with the raw EEG signals.

Once the raw EEG and noise signals are received and the noise signal is processed or pre-processed, the signal processor 49 performs a comparison or exclusion step 8, in which the processor removes or eliminates the portions of the raw EEG signal that are present in the noise signal. The removal of the noise signal results in a clean EEG signal, which is then stored and/or displayed to a user in step 10.

The signal processor 49 is preferably a digital processor, e.g., a computer, and the EEG signals and the noise data are electronic signals in the form of digital data that is processed by numerical wave-processing methods. However, the signal processing here described may be adapted to be used in a system in which the input raw EEG signals and noise layer signals are analog signals. In either case, however, the signal data is processed in real-time, or with a few milliseconds delay so that output or storage is essentially immediate.

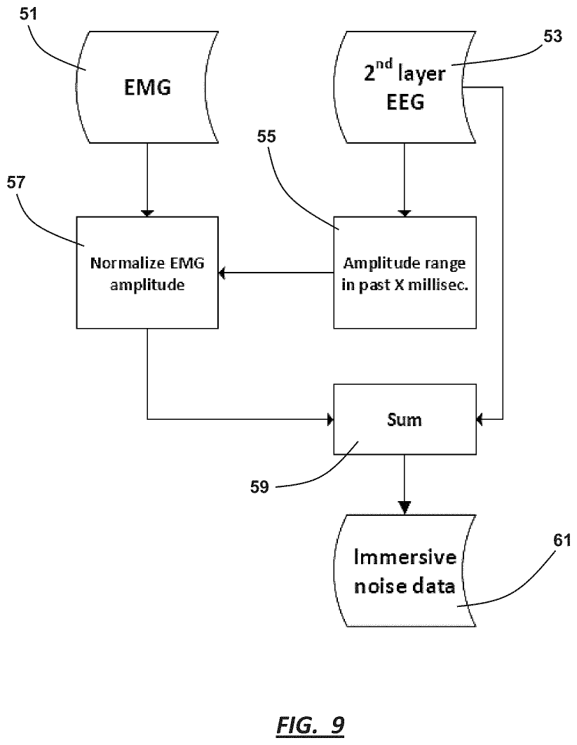

FIG. 9 shows in more detail a way in which the noise signal data is processed and derived in the preferred embodiments by combining the signals from EMG electrodes, which may be on the electrode headgear net or on other parts of the body of the subject, with the signals from the second noise layer, resulting in the derivation of data indicating a large part of the noise in the system.

Generally, EMG signals have a much higher amplitude than EEG signals, by a great deal. To adapt to this, the system receives the EMG signal 51 and the second layer noise signal 53, and scales down the EMG amplitude as set out in FIG. 9.

The EMG may be an analog signal if the EEG apparatus is one in which the EEG remains analog, or a digital one in which the EEG signal has been converted to digital, as in the preferred embodiment EEG apparatus described above. In either case, the second-layer noise signal is sampled for a period of milliseconds, e.g. 100 milliseconds or less, and the amplitude range during that period is determined (step 55). That amplitude range is then used to scale down the EMG output data and normalize it (step 57) so that it doesn't drown out all other data. The scaled-down EMG data is then summed with the second layer noise data (step 59) to yield immersive noise data 61, which contains essentially all of the environmental noise to which the EEG is subject. Merging the EMG in the noise-layer data after adjusting the EMG amplitude with the amplitude of the noise from the noise layer ensures that both can be identified during post-processing. That immersive noise data is supplied to the signal processor 49 for removal from the raw EEG signal (step 8 or FIG. 8).

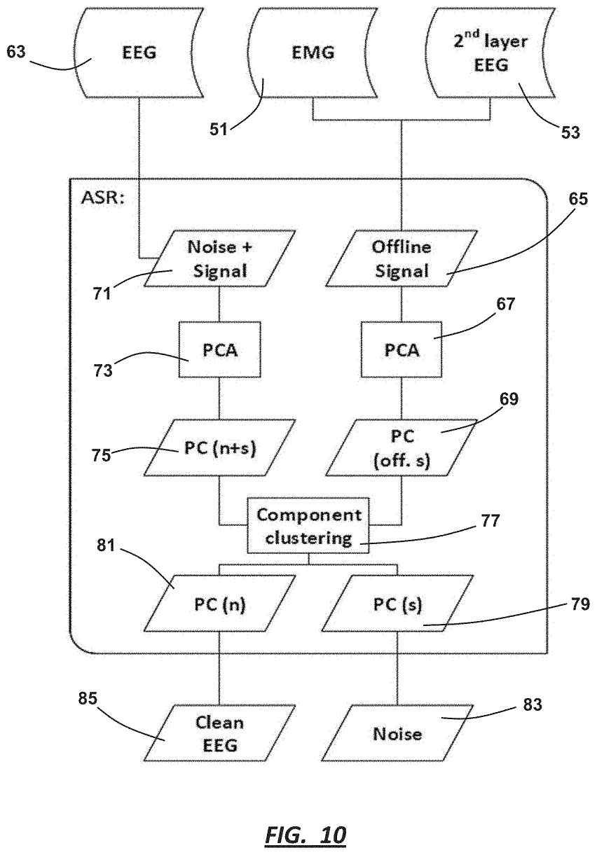

FIG. 10 shows the method as shown in FIG. 8 in greater detail, and in which cleaning up the noise in the EEG signal uses a reverse artifact subspace reconstruction (ASR) method for artifact separation. The data supplied to the EEG signal processor 49 is from the outputs of the signal hub 47 as individual signal streams for each electrode 3, and individual signal streams from outer-layer electrodes 13 and EMG electrodes.

The commonly used “forward” ASR method determines the components of the “noise-free” signal offline (i.e., before use of the EEG apparatus) using principal component analysis (PCA). The ASR then tries to find the components of the noise-plus-signal mixture during the EEG reading that have the same characteristics as the “noise-free” components that were figured out beforehand, and then outputs those components as the cleaned signal.

In contrast, the present method receives the pure noise and EMG from the immersive noise data as though it were the “noise-free” data, and then tries to find the EEG components that are the most similar to immersive noise data components. Those similar noise components are then rejected from the EEG electrode output, producing remaining EEG components that have minimized artifacts.

As shown in the diagram of FIG. 10, the EEG signals 63 from the first electrodes are transmitted to the EEG signal processor 49, together with the EMG signals 51 and the second layer outputs 53. The EMG signals are combined with the second layer signals as described above, yielding the immersive noise data signal.

The immersive noise data signal is then input to the ASR process as though it were the “noise-free” offline signal 65 input to the ASR in the prior art. In the present design, however, the pure immersive noise data signal, without the EEG signal, is inputted and subjected to PCA analysis (step 67) that converts the immersive noise signal data into data defining a set of principal components 69, i.e., constituent waveforms of the noise.

The EEG output 63 is from the inside electrodes 3, and is a combination of noise and the EEG biosignals 71. That signal is also subjected to PCA in real time (step 73) to yield data defining another set of principal components 75 containing components of both the noise and the desired EEG signals.

The data defining the two sets of principal components (i.e., the components of the EEG-plus-noise, and the components of the immersive noise alone) are then compared with each other in a component clustering step 77, in which the set of principal components are divided into data defining a set of the principal components found in both the EEG-plus-noise and immersive noise signals (79), and data defining the principal components found only in the EEG-plus-noise signals (81). The shared components are the noise part of the signals, and are output at step 83 as data for analysis, if desired. The components found exclusively in the outputs from the inner electrodes are the clean EEG signal, and are also output at 85 as the clean EEG signal. This corresponds to step 8 of the flowchart in FIG. 8. The clean EEG signal is then stored or displayed (step 10 of FIG. 8).

It should be noted that the PCA method is an exemplary method to decompose signals to its components. Other source-separation method including independent component analysis, canonical correlation analysis, empirical mode decomposition, variational autoencoders, singular value decomposition, and/or other artificial intelligence techniques can be used based on the use of the systems for the EXG applications.

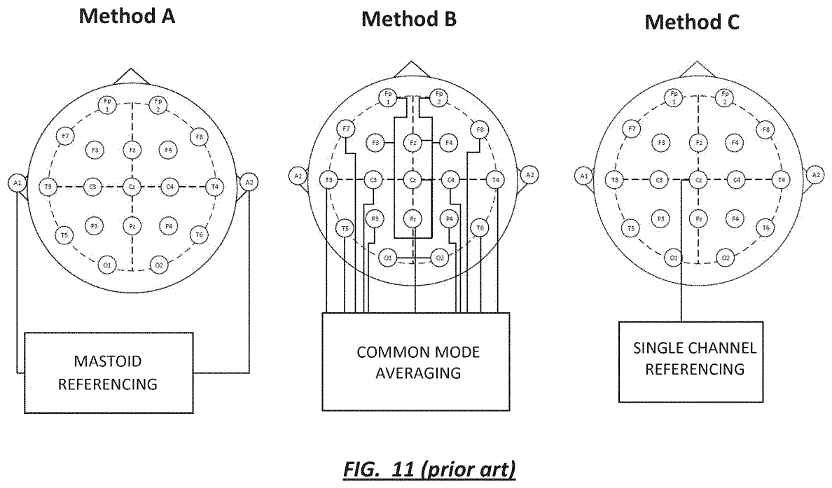

Three widely used EEG referencing methods of the prior art are shown in FIG. 11. In one, prior-art method A, the mastoid is assumed to be electrically neutral and inactive; either one mastoid electrode signal on the subject, or the average of electrode signals from both mastoid electrodes on the subject, is assumed as the reference. In prior-art method B, common-mode averaging, it is assumed that the universal electrical signals may originate from noise sources, and therefore, by referencing the average of all the electrodes (meaning the electrodes that are directed to the subject and detect biodata signals as well as noise signals), any large-effect of environment noise is rejected. In prior-art method C, using a single-channel of signal as reference, it is assumed that any electrical activity proximal to an EEG electrode is not critical in the analysis, so that activity can be assumed to be zero.

In all of these prior-art approaches, the reference signal is derived from the scalp, similar to EEG signals. Therefore, the EEG biodata signals are also partly canceled by use of a reference signal that is not electrically isolated from the EEG electrodes.

The determination of the noise layer signal to be removed from the raw EEG signal may be determined in a number of ways in the system of the invention.

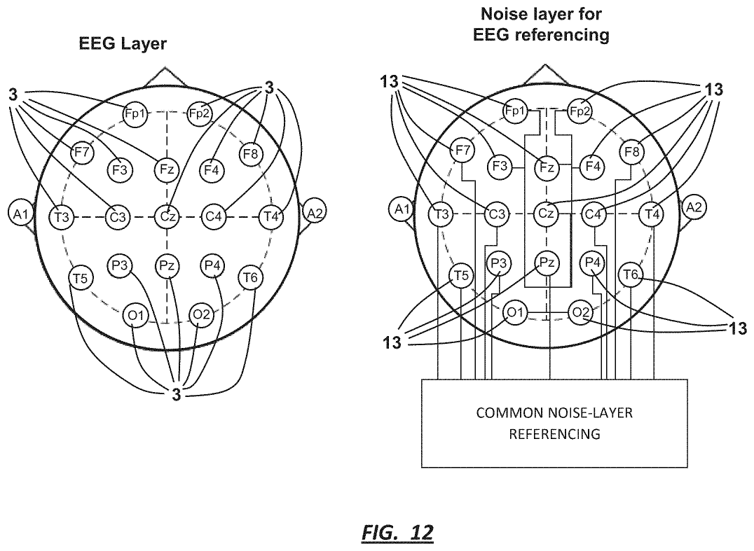

One method is illustrated in FIG. 12. In this method, the network of inner electrodes 3 all output respective output data signals for each of the EEG electrodes 3 sensing biosignals in the head of the subject, and the outer network of the outer electrodes 13 also all each produce respective outputs of each of the outer electrodes 13. According to the basic configuration of the system of FIG. 12, all of the outputs of electrodes 13 are combined and averaged to derive a single data signal that is considered the background noise level. Each of the actual detected EEG biosignals from electrodes 3 is then compared with that average noise signal as a reference (meaning that the reference signal is removed from the raw EEG signal, step 8 of FIG. 8) to derive a clean EEG signal for each electrode 3 relative to the average noise.

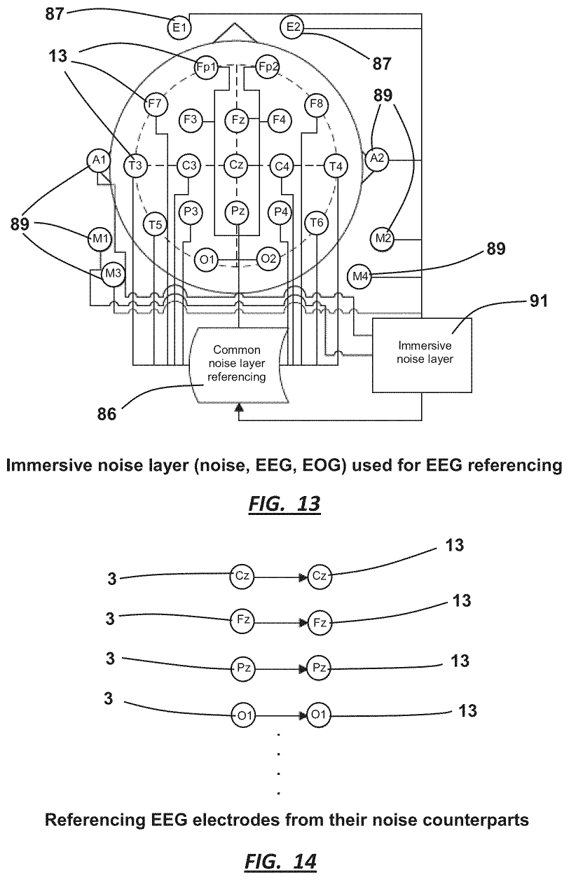

This average noise method can be improved by using the method of FIG. 13, in which the outputs of all the electrodes are combined and averaged, as in the previous method, to derive an outer-electrode noise signal average at 86. In addition, electrodes 87 and 89 sensing biosignals from the eyes, the mastoids and/or other muscular biosignals, i.e., EMG signals, are connected with the subject, and the output of those electrodes is scaled to the outputs of the outer electrodes 13 and combined (see FIG. 9) so as to produce an immersive noise layer signal 91. That noise signal 91 is combined or averaged with the averaged noise signal 86 to yield a noise reference signal to which every EEG signal 3 is compared as a reference (step 8 of FIG. 8) so as to remove the noise reference signal from the EEG signal and to derive the reduced noise EEG signal output for the respective electrode 3.

FIG. 14 illustrates another method of determining a reference signal for each electrode output 3. In this method, the output of each of the EEG electrodes 3 is compared with the output of the outer electrode 13 at that specific electrode unit (or node of the geodesic headgear) as its noise reference, and the noise reference signal is removed from the EEG electrode signal so as to yield a reduced-noise EEG signal. The resulting clean EEG signal is output for each electrode 3 on the subject, and stored and/or displayed, as described above.

This method may further be improved by first combining the noise signals of each outer-layer electrode 13 with a scaled immersive noise derived from electrodes sensing EMG, as described above in the method of FIG. 10, producing a more universal reference noise signal. The output of the inner electrode 3 associated with the outer electrode 13 is then compared with that reference noise signal, and the portion of the inner electrode output shared with it is removed or eliminated, producing a clean EEG biosignal.

Another method for determining the noise present in the system may use a trained or adaptive neural network, or a time-series generative neural network, such as a conditional time-series adversarial network (ctGAN), that is trained to identify and/or augment noise signals. In that context, training may take place by providing the GAN with a publicly available dataset of electrode outputs for EXG, and EEG in particular from public repositories such as openenuro.org, or eegnet.org to learn the features of “clean” EEG signals, including but not limited to learning the latent features of the signals to be used as templates for clustering of the components as discussed in 77, FIG. 10. Alternatively, the training may also be performed using the public EEG datasets mentioned above combined with artificially injected noise causing the GAN to repeatedly attempt to derive the noise signal in the output fed to it until a noise signal without any biosignal is derived. That trained GAN may then be used in the above-described reference signal derivation methods to derive a real-time immersive noise signal that is compared with, and eliminated from, the real-time raw EEG data signal, as has been discussed with respect to the other reference signal methods, and seen in e.g., FIG. 8. Adaptive neural network methods may be used to change the parameters of the trained network to adapt to the signals and noise being recorded. Artificial intelligence techniques such as super-resolution using convolutional, non-convolutional, or generative neural networks may be used to enhance the information contained in the immersive noise layer. The outputs of the neural networks may be used as the augmented noise signals, the reference for EEG signals, or the clustering parameters 77. In another setting, such methods may be used to reduce the number of noise electrodes 13, while maintaining the immersive noise data with artificial intelligence techniques. Therefore, the apparatus presented in the prior art, FIG. 2, and the headgear subject to this invention, FIG. 3, may be utilized with a subset of electrodes that only include the scalp-facing electrodes 3 and another subset of electrodes that comprised of both scalp-facing electrodes 3 and noise electrode 13. Subsequently, the artificial intelligence techniques discussed above reconstruct the signals of the missing noise electrodes from the remaining noise electrodes to create the immersive noise layer, the reference as discussed above in FIGS. 12 and 13, the input for the reverse ASR method in FIG. 10, or any combination of those. Reducing the number of electrodes may help with faster apparatus setup, lower data bandwidth, and less processing power.

Generally, the noise signal output derived by any of the above methods, i.e., the average of the second-layer electrode outputs, the combination of the second-layer outputs with EMG signals, and the use of the corresponding outer-layer electrode signal (with or without an EMG signal) is compared with and eliminated from the raw EEG signal from the inner layer electrode. This may be accomplished by using the EEG reference noise output from any of these methods as input to the reverse ASR method shown in FIG. 10. In that context, the prepared noise signal and the raw EEG signals are divided into their principal components, as described above, and then those component sets of the raw EEG and the processed noise are compared, with the shared components removed from the EEG signal, leaving the clean EEG for output or storage.

Although the use of EEG signal detection is customarily in the medical area, there are new applications for the use of EEG devices to which the present invention is applicable. In particular, virtual-reality headsets, augmented-reality gear, and wearables that have embedded biosignal sensors, including EEG electrodes, can derive a benefit from the noise cancellation systems described herein, and can as a result provide a direct brain-machine interface in the headset.

The terms used herein should be understood to be terms of description rather than limitation, as those of skill in the art with this disclosure before them will be able to make modifications in the disclosed system without departing from the spirit of the invention.

What is claimed is:

1. An apparatus for sensing biosignals of a head of a subject, said apparatus comprising:
a net structure configured to be supported on the head of the subject;
the net structure comprising a plurality of electrode structures connected in the net structure by elastic legs each connected with a respective pair of the electrode structures;
the electrode structures each including
a respective first electrode directed toward and sensing biosignals in the head of the subject;
a respective second electrode supported adjacent the first electrode and directed away from the head of the subject and sensing electrical signals in an environment around the head of the subject;
the legs each having
a respective elastic conduction element extending between the associated electrode structures, the conduction elements being connected electrically with the second electrodes of the electrode structures connected with the leg; and
a respective elastic insulation structure between the associated conduction element and the head of the user so as to electrically insulate the conduction element from the head of the user.

2. The apparatus of claim 1, wherein the legs also each have an outwardly disposed elastic insulation layer outward of the elastic conduction elements.

3. The apparatus of claim 1, wherein the net structure is an arrangement in which each of the electrode elements is connected with five or six of the legs, all of said legs having the conduction elements thereof connected with the second electrodes so that the second electrodes of the net structure are all interconnected electrically, and wherein the net structure has electrical properties that are similar to electrical properties of the head of the user.

4. The apparatus of claim 3, wherein the net structure includes further electrode elements having first and second electrodes at a perimeter of the net structure, said further electrode elements having four or fewer links to adjacent electrode structures of the net structure.

5. The apparatus of claim 1, wherein the electrode structures each have a respective analog to digital converter receiving electrical signals from the first and second electrodes and converting said electrical signals to digital signals that are output to digital circuitry that processes the digital signals so as to derive EEG data therefrom.

6. The apparatus of claim 5, wherein the electrode structures each have a multiplexer receiving raw signals from the first and second electrodes and multiplexing said raw signals with a control signal having a frequency of 5 kHz or greater and transmitting a resulting multiplexed output to the analog/digital converter.

7. The apparatus of claim 5, wherein the digital circuitry processes the digital signals by identifying noise in the signals from the second electrodes, and then producing EEG signals derived from the signals from the first electrodes from which the noise is removed.

8. The system of claim 7, wherein the identifying of the noise includes separating the signals into component waveforms, averaging the signals from the second electrodes, or processing the signals of the second electrodes with a neural network trained to identify the noise of the net structure.

9. The apparatus of claim 8, wherein the system further comprises additional electrodes generating signals responsive to muscle activity of the subject, and
wherein the identifying of the noise includes averaging the signals from the second electrodes and the signals of the additional electrodes prior to removing the noise from the signals from the first electrodes.

10. A method of sensing electrical currents in skin of a subject, said method comprising:
deriving an output from a first electrode directed toward the skin of the subject;
deriving an output from a second electrode connected with the first electrode and directed away from the skin of the subject in an electrically connected net structure that has electrical properties similar to electrical properties of the skin of the subject;
determining a noise component in the signal from the second electrode; and
storing or outputting EEG data derived from the output from the first electrode from which the noise component has been removed.

11. The method of claim 10, wherein the determining of the noise component includes dividing the output from the second electrode into a set of discrete waveform components; and
wherein the EEG data is derived by dividing the output from the first electrode into a respective set of discrete waveform components, and then clustering the discrete waveform components so as to identify the discrete waveform components that are present in the output from the first electrode but not in the output from the second electrode, and removing from the output of the first electrode the waveform components that are present in the output of the second electrode.

12. The method of claim 11, wherein the method further comprises
deriving additional signals from additional electrodes picking up electrical background currents created by muscle activity in the subject; and
scaling the additional signals to correspond in amplitude to amplitudes of the signals from the second electrodes over a period of a number of milliseconds prior thereto; and
wherein the determining of the noise includes combining the scaled additional signals with the signals from the second electrodes.

13. The method of claim 12, wherein the additional electrodes are operatively associated with a mastoid, an eye or a muscle of the subject; and
wherein the scaling includes determining an amplitude range of the output from the second electrode over a predetermined period of time, scaling an amplitude of the output of the additional electrode to correspond to the amplitude range of the output of the second electrode, and then summing the scaled output with the output of the second electrode to determine said noise.

14. The method of claim 10, wherein the skin of the subject is on a head of the subject on which the first electrodes are placed with conductive gel therebetween.

15. An apparatus for sensing biosignals of a head of a subject, said apparatus comprising:
a structure configured to be supported on the head of the subject;
the structure comprising a plurality of electrode structures;
the electrode structures each including
a respective first electrode directed toward and sensing biosignals in the head of the subject;
a respective second electrode supported adjacent the first electrode and directed away from the head of the subject and sensing electrical signals in an environment around the head of the subject;
the electrode structures having electronic circuitry therein that receives the outputs of the first and second electrodes, converts the outputs to digital signals in the electrode structure, and transmits the digital signals to a signal processor external to the head mounted structure.

16. The apparatus of claim 15, wherein the electronic circuitry includes a multiplexer that combines the signals so as to form a single electrode output signal, and an analog/digital converter that converts the single electrode output signal to a sequence of digital data signals with a voltage of 2 to 6 volts each corresponding to the amplitude of the signal from one of the electrodes, and transmits the sequence of the digital data signals along a single conductor to the signal processor.

17. The apparatus of claim 16, wherein the multiplexer multiplexes the digital signals by outputting the single electrode output signal for a cycle of a control signal as the output of the first electrode, and then switching in a next cycle of the control signal to output the single electrode output signal for the next cycle of the control signal as the output of the second electrode, and then switching back to the output of the first electrode so that the single electrode output signal alternates between the output of the first electrode and the output of the second electrode every cycle of the control signal.

18. The apparatus of claim 17, wherein the control signal has a frequency of at least 3 kHz.
